(19) United States
(12) Patent Application Publication
(10) Pub. No.: US 2019/0192813 A1
(43) Pub. Date: Jun. 27, 2019

(54) SECONDARY LUMEN FOR RAPID DRUG INFUSION

(71) Applicant: Atsuko NAKAGAMI CETL, São José dos Campos (BR)

(72) Inventors: Atsuko NAKAGAMI CETL, São José dos Campos (BR); Luiz Daniel MARQUES NEVES CETL, São José dos Campos (BR)

(21) Appl. No.: 16/168,284

(22) Filed: Oct. 23, 2018

(30) Foreign Application Priority Data
Dec. 22, 2017 (BR) 10 2017 027860 3

(51) Int. Cl. A61M 25/00

(52) U.S. Cl. CPC A61M 25/0026; A61M 25/01

(57) ABSTRACT

A secondary lumen for rapid drug infusion in association with a double, triple or multiple lumen catheter, wherein, notably, said secondary lumen (100) consists of a bore (101), with one end equipped with a connector (102) and the opposite end in the form of a hollow tip (103); the bore (101) has an inner diameter (d1) that enables the passage of a liquid drug (A) and an outer diameter (d2) inferior to the diameter (d3) of at least one of the lumens (L1) of a tube (T1) of a double, triple or multiple lumen central venous catheter (VC), such that said bore (101), when inserted in the lumen (L1) of the venous catheter (VC), has the hollow end (103) coinciding with the hollow end (V1) of the said lumen (L1) of the tube (T1) and has part of the bore (101) of the secondary lumen and the respective connector (102) facing away from the venous catheter (VC).

### FIELD OF INVENTION

The present invention refers to a secondary lumen for rapid drug infusion in association with a double, triple or multiple lumen catheter, wherein, notably, said secondary lumen is specially positioned in the catheter in a way to receive a vasoactive drug, particularly of the noradrenaline type and correlates, and to administer it directly in the intensive treatment environment with full control of the dose and time of administration, thereby ensuring balanced, proportional, rapid and efficient administration, especially in moments of shock, cardiac arrest and acute hypertension.

### BACKGROUND OF THE INVENTION

It is known that the market of medical products has various models of central venous catheters (CVC), and among its main models are those used for hemodialysis, checking central nervous pressure, drawing blood and administering drugs. There are a number of models with characteristics that vary according to the manufacturers and their function. The central venous model is inserted percutaneously in central internal jugular, femoral or subclavian veins.

Generally, the central venous catheter (CVC) consists of a flexible tube structure made of either silicone or polyurethane equipped with a connector for mounting one or various lumens, which are of varied diameters and classified according to the proximal or distal distance to the heart, where the determination of the catheter material and number of lumens depend on the necessities of the patient and the therapeutic objectives.

However, the manipulation of the central venous catheter is not free from complications or risks like phlebitis, vein thrombosis, arrhythmia, embolism, site infection, sepsis, endocarditis, displacement of the catheter or migration of catheter tip, clot formation and lumen obstruction, among others, although some complications can be prevented by respecting the aseptic technique in the procedures.

Some complications are intrinsic to the administration of the drug, for example, during the control of noradrenaline administration, which is a drug of choice in septic shock and whose purpose is to increase the blood pressure of hypotensive patients who do not respond to resuscitation by volume and to other less potent inotropic drugs. Noradrenaline is also always used as a vasoconstrictor drug during cardiopulmonary resuscitation (CPR) maneuvers.

In case of noradrenaline, the professionals use under normal working conditions, 1 to 4 ampoules of 2 mg diluted in 250 ml of any solution normally used, aiming to infuse a final concentration of 1 μg/min. The infusion must be administered in a large vein and the infusion site must be changed at least every twelve hours.

However, when there is a situation of stress, such as an emergency, or shock, it is common for the nurse to resort to the insertion of a double lumen catheter applied in the central artery so as to administer the drug as close to the heart of the patient as possible in order for the drug to act rapidly. In such cases, noradrenaline must also be administered urgently due to the changes in the patient's blood pressure. However, due to the infusion procedure of the drug in these cases, depending on the mounting of the solution in the device and in the double lumen catheter, the physical distance of the fluid trajectory between the solution outlet and entry close to the heart is considered too great, especially considering that the flow of noradrenaline must be 3 ml/hour.

Therefore, it is common during times of stress for the nurse to completely open the flow of the solution in order to conduct the noradrenaline to the heart as fast as possible, causing a peak in pressure, which then returns slightly, allowing the flow of the solution to be reduced.

This inaccuracy during the administration of this type of drug can cause several side effects, such as a CVA in the patient, or even internal bleeding, among other problems resulting from these peaks in pressure.

During research that was conducted in a specialized database, several documents were found on central venous catheters with single or multiple lumens, such as document US2008221509, which refers to a catheter with tubes that extend into lumens that can be used for blood or drugs, and can also be used for sensors.

Another document U.S. Pat. No. 4,721,115 refers to a catheter with various lumens that have a balloon-like expander located at its distal end, and the inside of the balloon is connected to single or multiple lumens through which a fluid introduced in the proximal end of the catheter can be used to inflate the balloon.

Other documents related to the catheter with single or multiple lumens were located, such as documents U.S. Pat. No. 5,250,038, US2006004325, U.S. Pat. No. 5,000,190 and U.S. Pat. No. 5,797,869, but despite the fact that these belong to the same field of application of the object in question, they differ from the present application and, therefore, are not prior art in relation to the improved object, thus ensuring its compliance with the legal requirements of patentability.

It was observed that the current arrangements of the conventional catheters with double, triple or multiple lumens prevent the patient in shock from immediately receiving the said drug, since the length of the main lumen to the distal outlet prevents the drug from being received instantly, a fact that may compromise the recovery of the patient.

### SUMMARY OF THE INVENTION

The present invention seeks to provide a secondary lumen for rapid drug infusion, particularly vasoactive drugs like noradrenaline or other drugs, in association with the catheter with double, triple or multiple lumens, allowing, with the insertion of the innovative secondary lumen in the said catheter, the infusion of the drug in the right, balanced and objective dosage, in other words, closest to the distal outlet of the lumen.

The present invention also seeks to provide for the drug to be administered in the desired time with the use of the secondary lumen.

The present invention also seeks to provide a secondary lumen where the infusion of the drug in the ideal proportion aims to neutralize the risks of overdose, as it occurs nowadays.

The present invention also seeks to provide a low cost secondary lumen, ensuring the use by any public or private hospital network.

### BRIEF DESCRIPTION OF THE DRAWINGS

FIG. 1 represents an exploded perspective view illustrating the position of the secondary lumen in relation to a double-lumen type venous catheter model.

FIG. 2 shows the same perspective view of the previous view, illustrating the secondary lumen of the invention introduced into one of the lumens of the venous catheter, showing its position of use on the patient.

FIG. 3 illustrates an expanded detail of the end of the double-lumen venous catheter with the secondary lumen in its final position, which is for infusion of the drug in the patient.

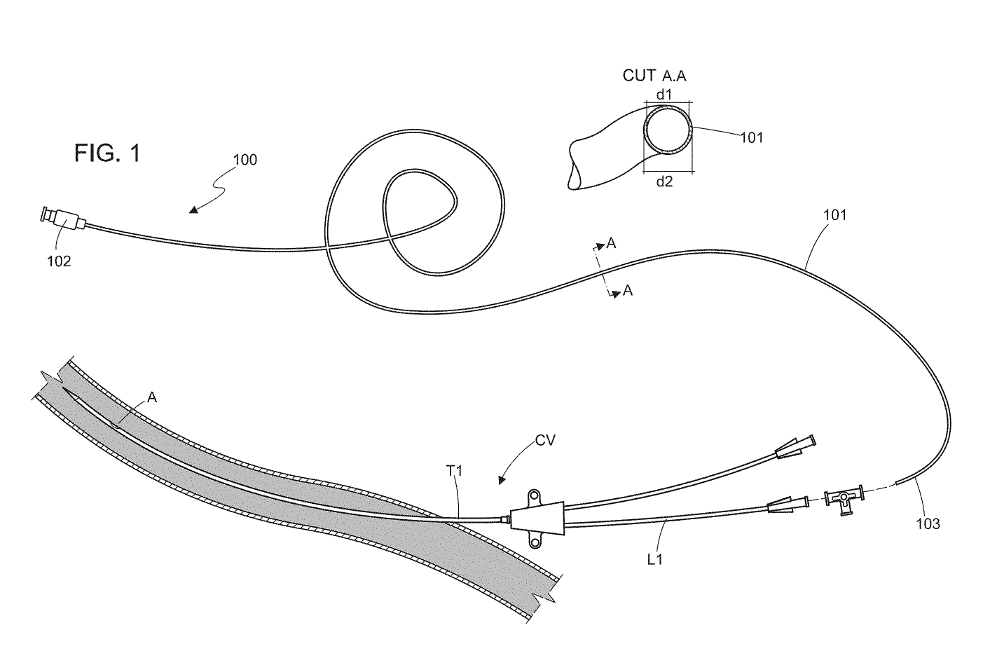

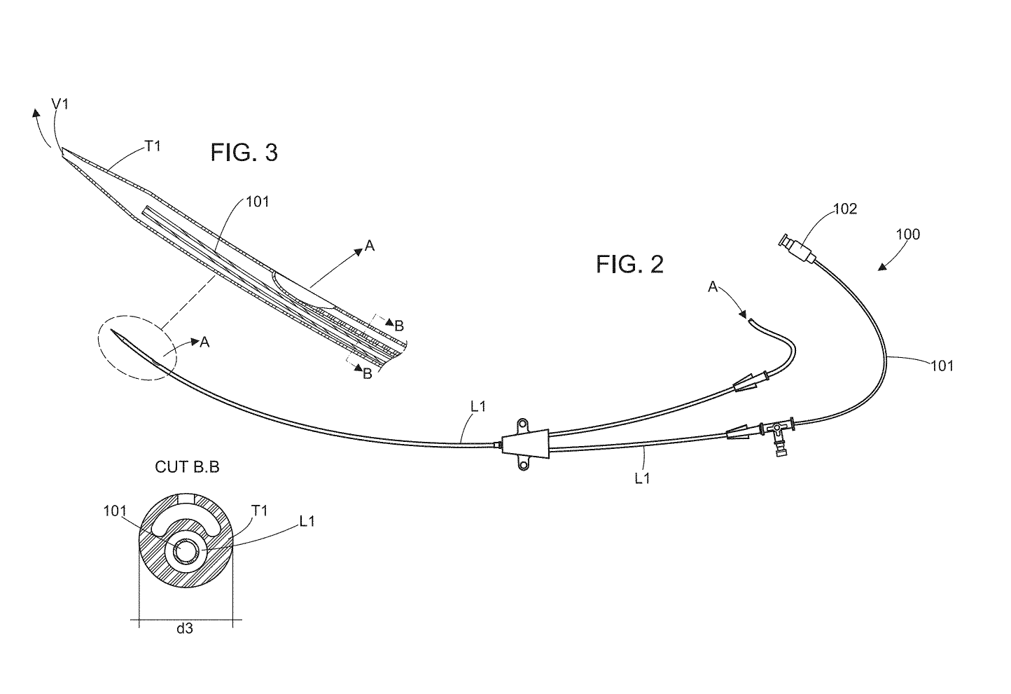

### DETAILED DESCRIPTION OF THE INVENTION

The present invention refers to a secondary lumen for rapid drug infusion. More precisely, it refers to a secondary lumen (100) consisting of a bore (101), with one end equipped with a connector (102) and the opposite end in the form of a hollow tip (103); the bore (101) has an inner diameter (d1) that enables the passage of a liquid drug (A) and an outer diameter (d2) inferior to the diameter (d3) of at least one of the lumens (L1) of the tube (T1) of a double, triple or multiple lumen central venous catheter (VC), such that said bore (101), when inserted in the lumen (L1) of the venous catheter (VC), has the hollow end (103) coinciding with the hollow end (V1) of the said lumen (L1) of the tube (T1) and has part of the bore (101) of the secondary lumen and the respective connector (102) facing away from the venous catheter (VC).

In one embodiment, the secondary lumen (100) has a length equivalent to or greater than the length of the double, triple or multiple lumen central venous catheter (VC).

In another embodiment, the secondary lumen (100) may be supplied with a dose (ds) of drug in the bore (101) in completely sterile conditions and in the appropriate quantity for injection in the central artery (CA) of the patient (not illustrated).

In another embodiment, the connector (102) can be coupled directly to the infusion pump (not illustrated) to aid the flow of the infusion drug in the secondary lumen (100).

It is certain that when the present invention is put into practice, modifications may be introduced in relation to certain details of construction and shape, without compromising the fundamental principles that are clearly substantiated in the claims table, thus being understood that the terminology used had no limiting purpose.

1. A secondary lumen for rapid drug infusion comprising:
a bore, with one end equipped with a connector and an opposite end in the form of a hollow tip; the bore having an inner diameter enabling the passage of a liquid drug; and an outer diameter suitable for connecting to at least one hollow end of a lumen of a tube of a central venous catheter,
wherein, when the bore is inserted in the lumen of the central venous catheter, the hollow tip coincides with the hollow end of the lumen of the tube, and
wherein a section of the bore of the secondary lumen and the connector are facing away from the central venous catheter.

2. The secondary lumen according to claim 1, wherein the secondary lumen has a length equivalent to or greater than the length of the central venous catheter.

3. The secondary lumen according to claim 1 wherein the secondary lumen is supplied with a dose of the liquid drug in the bore in completely sterile conditions and in an appropriate quantity for injection in the central artery of a patient.
